(19) United States
(12) Patent Application Publication
(10) Pub. No.: US 2009/0005734 A1
(43) Pub. Date: Jan. 1, 2009

(54) Systems and methods for delivering particles into patient body

(75) Inventors: Mathieu Herbette, Mountain View, CA (US); Curtis Tom, San Mateo, CA (US)

Correspondence Address: PATENT OFFICE OF DR. CHUNG S. PARK, P. O. BOX 62312, SUNNYVALE, CA, 94088-2312, US

(73) Assignee: Tessaron Medical, Inc., Sunnyvale, CA (US)

(21) Appl. No.: 11/823,380

(22) Filed: Jun. 27, 2007

(51) Int. Cl. A61M 5/44; A61M 25/01; A61M 39/22; A61M 5/142; A61M 5/178; A61M 5/32

(52) U.S. Cl. 604/113; 604/151; 604/187; 604/246; 604/256; 604/272; 604/528

(57) ABSTRACT

Apparatus and methods for delivering particles to a target region in a patient's body. The apparatus includes a hub and a catheter having a proximal end coupled to and in fluid communication with the hub. The distal end of the catheter is inserted into the target region. The apparatus also includes: means for directing substrate to the target region, the means being coupled to the hub and in fluid communication with the catheter; a pressure sensor for measuring a pressure of the substrate and generating a signal commensurate with the pressure; and a pressure display for displaying the pressure in response to the signal.

### BACKGROUND

The present disclosure generally relates to medical methods and apparatus, more particularly, to delivering particles into a patient body for thermal treatment.

Human and/or animal can suffer from various types of tissue-related illnesses, such as breast cancer and incontinence. Breast cancer may be the most common cancer that forms in tissues of the breast, usually the ducts (tubes that carry milk to the nipple) and lobules (glands that make milk). In general, breast cancer has two types: in situ and invasive. In situ breast cancer is a type of cancer in which the breast cancer cells have remained contained within their place of origin, i.e., they haven't invaded breast tissue around the duct or lobule. Invasive (infiltrating) breast cancers are those that break free of where they originate, invading the surrounding tissues that support the ducts and lobules of the breast. In some cases, the cancer cells can travel to other parts of the body, such as the lymph nodes.

Incontinence, which refers to involuntary urination, is experienced by older adults who have difficulty with bladder control usually because of either urinary tract disease, nervous system dysfunction, allergic response, ruptured disk, or psychological stress. Women tend to experience involuntary urination after childbirth, surgery, or inflammation of the urethra, while men tend to get it if they have a prostate problem.

Various types of techniques have been developed to treat abnormal tissue. For instance, one technique to treat breast cancer may be removal of the affected duct or the entire portion of the breast to provide the best assurance against recurrence of the cancer, but may be disfiguring and require the patient to make a very difficult choice and, quite often, to have a subsequent cosmetic surgery. (Hereinafter, the term cancer collectively refers to cancerous, pre-cancerous, and other abnormal cells or disease conditions.) Chemotherapy and radiation can be another technique, but cannot provide an effective assurance against recurrence. Lumpectomy can be an alternative approach, but is associated with a substantive chance of recurrent. For another instance, homeopathic treatment may be the most common approach to relieve incontinence, but does not solve the fundamental problem of the incontinence.

### SUMMARY OF THE DISCLOSURE

In one embodiment, a method for delivering particles to a target region in a patient body via a catheter includes steps of: inserting a distal end portion of the catheter into the target region; directing a first substrate through the catheter to the target region; drawing the first substrate from the target region through the catheter; and directing a second substrate containing the particles through the catheter to the target region.

In another embodiment, an apparatus for delivering substrate to a target region in a patient body includes: a hub; a catheter having a proximal end coupled to and in fluid communication with the hub and a distal end to be inserted into the target region; means for directing substrate to the target region, the means being coupled to the hub and in fluid communication with the catheter; a pressure sensor for measuring a pressure of the substrate and generating a signal commensurate with the pressure; and a pressure display for displaying the pressure in response to the signal.

### BRIEF DESCRIPTION OF THE DRAWINGS

FIG. 1 shows a schematic diagram of a device for delivering particles into a patient body in accordance with one embodiment of the present invention;

FIG. 2 shows a schematic functional block diagram of a system to inject particles into a patient body using the device in FIG. 1;

FIG. 3A shows a schematic longitudinal cross sectional view of one embodiment of a catheter;

FIGS. 3B and 3C show schematic longitudinal and transverse cross sectional views of another embodiment of a catheter;

FIGS. 3D and 3E show schematic longitudinal and transverse cross sectional views of yet another embodiment of a catheter; and

FIG. 3F shows a schematic longitudinal cross sectional view of still another embodiment of a catheter.

### DETAILED DESCRIPTION OF THE PREFERRED EMBODIMENTS

The following detailed description is of the best currently contemplated modes of carrying out the invention. The description is not to be taken in a limiting sense, but is made merely for the purpose of illustrating the general principles of the invention because the scope of the invention is best defined by the appended claims.

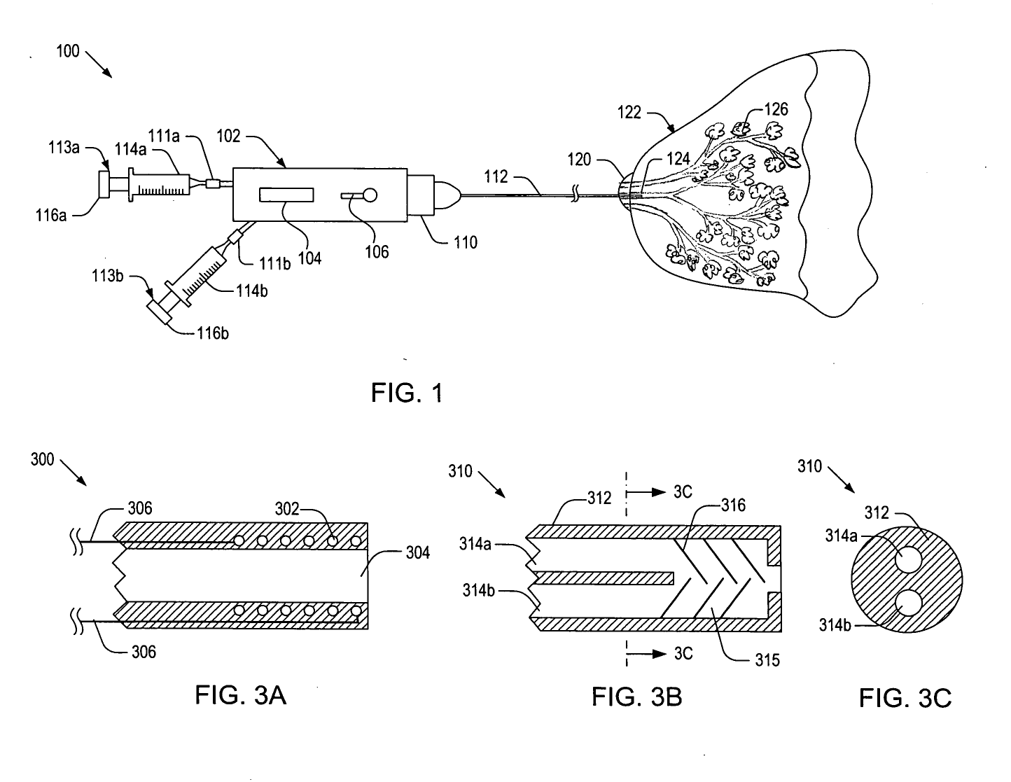

Various types of diseased/malignant cells can be treated by a hyperthermia technique. In the hyperthermia treatment, particles that generate beneficial heat energy upon excitation by an external electromagnetic field are delivered to the target tissue. The target tissue may be treated by elevating the temperature of its individual cells to a lethal level. FIG.1 shows a schematic diagram of a device 100 for delivering the particles into a human breast 122 in accordance with one embodiment of the present invention. Hereinafter, for the purpose of illustration, a breast cancer is shown as exemplary target tissue. However, it should be apparent to those of ordinary skill that the device 100 can be used to deliver particles to other types of diseased and/or malignant tissue as well as blood vessels, such as varicose veins to be closed off by the hyperthermia treatment. As depicted, a human breast 122 has a nipple 120; and ductal networks 124 that extend inwardly from the nipple and then into branching networks. Each network 124 includes a series of successively smaller lumens which are arranged in three dimensional configurations. Attached to the end of the smallest lumen is a lobule 126 for generating milk.

One type of breast cancer is Invasive Ductal Carcinoma (IDC). IDC accounts for about 80% of all breast cancers. Invasive means that it has “invaded” or spread to the surrounding tissues. It is ductal because the cancer began in the milk ducts—which are the “pipes” that bring milk from the lobule 126 to the nipple 120. Carcinoma refers to any cancer that begins in the skin or other tissues that cover internal organs—such as breast tissue. Another type of breast cancer is Invasive Lobular Carcinoma (ILC). ILC accounts for about 10%-15% of all breast cancers. It is lobular because the cancer began in the lobules 126. In both IDC and ILC cases, if the cancer cells spread around a network, the entire network may be considered as target tissue (or, equivalently, target region) that needs proper treatment thereby to reduce the chance of recurrence.

The particles may include material that can generate beneficial heat energy in response to an alternating electromagnetic field external to the breast 122. Systems and methods for exciting the particles are disclosed in U.S. patent application Ser. No. ______, entitled “Systems And Methods For Inductive Heat Treatment Of Body Tissue,” filed on Jun. 27, 2007, which is herein incorporated by reference in its entirety.

The size of the particles may be determined by the target tissue to be treated. For instance, the dimension of the particles for breast cancer treatment may be smaller than the finest duct diameter such that the particles may reach the lobules 126. Other known types of particles, such as particles having a coating, may be injected into the network 124 by the device 100.

The device 100 may include a hub 102 having one or more ports 111a, 111b for respectively coupling to syringes 113a, 113b. Each syringe may contain a selected drug, surfactant, medium carrying particles, liquid, or the like in a predetermined amount that is to be injected into the network 124. A syringe, say 113a, may include a cylinder 114a in which a plunger 116a is reciprocatingly disposed. The plunger 116 may be operated by a physician or coupled to a pump motor for reciprocating the plunger. As a variation, a container or reservoir may be fluidly coupled to each syringe to provide a content to the syringe. Alternatively, a container or reservoir with a suitable plunger or piston mechanism may be used in place of or coupled to each syringe. For simplicity, only two ports 111a, 111b are shown in FIG. 1. However, it should be apparent to those of ordinary skill that other suitable number of ports may be installed in the hub 102 without deviating from the scope and spirit of the present teachings.

The hub 102 may include a pressure sensor (not shown in FIG. 1) for measuring the pressure in the network 124 and a display 104 for displaying the measured pressure. The hub 102 may also include: a safety pressure valve (not shown in FIG. 1) to prevent the network 124 from being overloaded by the plungers 116a, 116b; and a selection valve 106 that permits a user to select one of the syringes 113a, 113b. The safety pressure valve may be set to prevent the pressure in the network 124 from increasing beyond a preset level. It is noted that the hub 102 may include more than two ports and the selection valve 106 may allow a user to select more than one syringe simultaneously such that the contents in the selected syringes can be mixed during injection into the network 124. As a variation, the hub 102 may include multiple selection valves and each selection valve controls the flow of contents from a corresponding syringe.

The device 100 may include a catheter 112 having a proximal end coupled to the hub and a distal end to be inserted into a patient body, in this case, breast. The device 100 may also include a steering control 110 for steering the tip or distal end of the catheter 112. Certain embodiments of the catheter 112 may have a mechanism to steer the distal end portion thereof so that the practitioner can advance the catheter 112 into intended branches or locations within the patient body. The display window 104 may be preferably, but not limited to, an LCD window, and can display various items, such as pressure, solution selected by the valve 106, or the like.

To fill the particles in the network 124, a user/physician may insert the distal end portion of the catheter 112 into the ductal network 124 through the nipple 120 using minimally invasive techniques and subsequently a solution or substrate containing the particles may be injected into the network via the catheter 112. In one exemplary embodiment, the solution may be a surfactant having coverage promoting characteristics. The solution may reduce the surface tension of the fluid within the duct and the substrate carrying the particles. The solution may reduce the attraction between polar molecules and therefore permit the particles to not clump, which allows the particles within the substrate to flow into the finer branches of the ducts. The breast ductal network 124 has many branches and filling the target ductal network 124 with the particles thoroughly may be important for the success of the hyperthermia treatment. To ensure maximum coverage and to promote filling the finer branches and lobules in the ductal network 124, one or more pressure swing cycles may be repeated by use of the device 100.

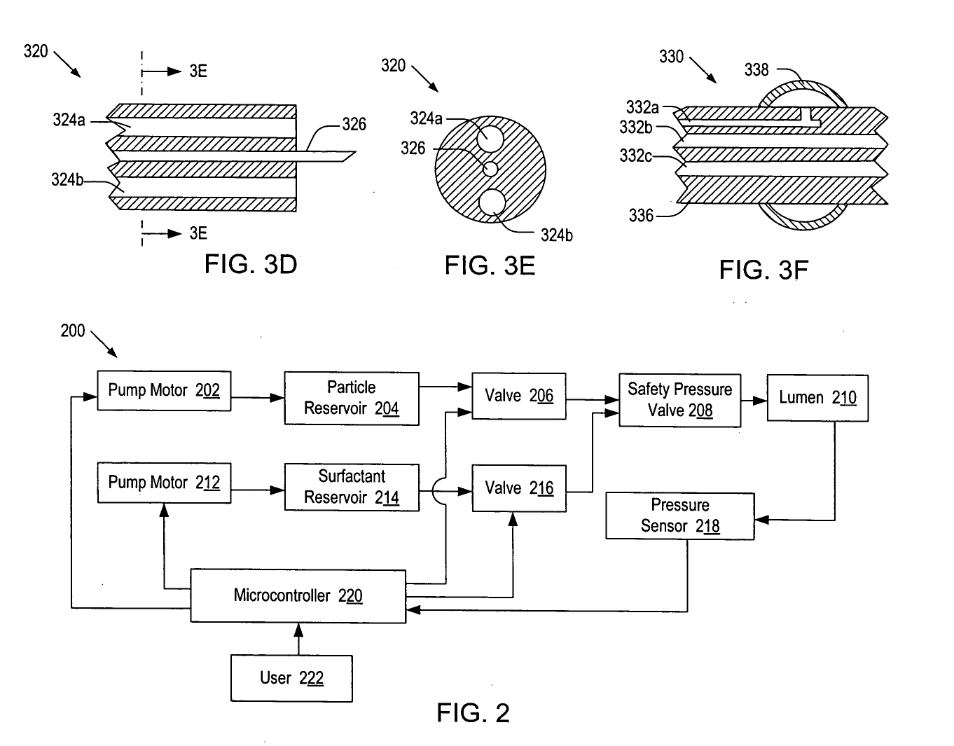

FIG. 2 shows a schematic functional block diagram of a system to inject particles into a patient body using the device 100 in FIG. 1. As depicted in FIG. 2, a user/physician 222 may operate a microcontroller 220 that can be programmed to operate the device 100. In one exemplary embodiment, the microcontroller 220 may be included in a computer (not shown in FIG. 2) that is coupled to the device 100. In another exemplary embodiment, the microcontroller 220 may be included in the hub 102 with a suitable user interface, such as control buttons, for providing input information to the microcontroller.

The user 222 may operate the microcontroller 220 to send a signal to a pump motor 202 so that the pump motor 202 can draw the particle containing solution from the particle reservoir 204 into the hub 102. In one exemplary embodiment, the particle reservoir may include the cylinder 114a and the pump motor 202 may depress the plunger 116a. In another exemplary embodiment, the particle reservoir 204 may be fluidly connected to the syringe 113a and the pump motor 202 may operate the plunger 116a to draw the particle containing solution from the reservoir and direct the solution into the hub 102.

The particle containing solution (or, shortly, particle solution hereinafter) drawn into the hub 102 may pass through a valve 206, which may correspond to the selection valve 106 (FIG. 1), and a safety pressure valve 208 to enter a lumen 210 formed in the catheter 112. The safety pressure valve 208, which may be mounted in the hub 102, may be responsive to the pressure of the solution in the lumen 210 or hub 102 to prevent tissue damage due to inadvertent overpressure. A pressure sensor 218, which may be included in the hub 102, may measure the pressure of the solution and send a signal commensurate with the pressure to the microcontroller 220. The microcontroller 220 may take the signal as one of the input signals to operate the pump motor 202, forming a closed loop control system with the pressure sensor 218.

When the pressure of the particle solution reaches a preset upper level, the microcontroller 220 may actuate the pump motor 202 to retract the plunger 116a so as to draw the injected solution from the ductal network until a pressure reaches a preset lower level, completing a pressure swing cycle. The pressure swing cycle may be repeated as intended and followed by filling the particle solution in the ductal network 124 so that the particles are thoroughly filled in the finer branches and lobules in the ductal network. In an alternative embodiment, the hub 102 may have another port coupled to a suction mechanism, such as vacuum pump, and the catheter 112 may have a lumen formed therein along the catheter length. In this embodiment, the microcontroller 220 may actuate the suction mechanism to draw the injected solution from the ductal network through the lumen in each pressure swing cycle.

The catheter 112 may incorporate a sealing mechanism, such as a balloon or a resolvable bioabsorbable plug to prevent the particle solution from leaking or migrating away from the target ductal network during the pressure swing cycles and/or hyperthermia treatment. Further detailed description of the sealing mechanism will be discussed in conjunction with FIG. 3F.

To enhance filling the particle solution in the ductal network 124, the duct wall may be coated with chemical, such as surfactant, prior to the injection of the solution. The surfactant may include saline solution, monoglycerides, phospholipids, or the like and may lower the surface tension of the duct wall. In one exemplary embodiment, a syringe, say 113b, may contain the surfactant, i.e., the syringe 113b may correspond to the surfactant reservoir 214. A plunger 116b may be reciprocatingly mounted in the cylinder 114b and operated by a pump motor 212 (FIG. 2). In another exemplary embodiment, a surfactant reservoir may be fluidly connected to the syringe 113b to provide the surfactant to the syringe. In both embodiments, the surfactant may be injected into the ductal network in the same manner as the particle solution, i.e., the closed loop system including the microcontroller 220, motor 212, and pressure sensor 218 may be used to inject the surfactant. Also, to promote uniform coating of the surfactant, one or more pressure swing cycles with the surfactant may be repeated.

In one exemplary embodiment, the pressure swing cycles for coating the surfactant and/or injecting particle solution in the ductal network 124 may be performed manually. For instance, the plungers 116a, 116b may be operated by a physician while the signal from the pressure sensor 218 may be displayed on a display window 104 to aid the physician in applying a proper force on the plungers. In another exemplary embodiment, the pressure swing cycles may be performed semi-automatically, i.e., the pressure swing cycles may be performed partly by the microcontroller 220 and partly by the physician.

In treatment procedures, such as a treatment of incontinence or small tumor ablation, the particles may be injected into a target tissue mass by a needle (FIG. 3D) and excited by external electromagnetic field to generate heat energy. A patient with incontinence loses urine involuntarily during physical activities that put pressure on the abdomen. The target tissue/muscle that does not close properly, such as weakened sphincter, bladder neck, or urethra, can be heated by the particles to shrink to an intended size such that the target tissue can restore urinary control. Desirable characteristics of incontinence treatment may include precise dosage of the particles and ability to immobilize the particles in the target zone. Immobilization of the particles may be required in other types of treatments, such as varicose veins and arteriovenous malformation (AVM). For instance, a varicose vein may be closed off by use of the heat energy generated by exciting the particles localized and immobilized in the vein. In one embodiment, the particles may be mixed with a highly viscous material, such as gel like medium, so that the injected particles are immobilized during treatment.

FIG. 3A shows a schematic longitudinal cross sectional view of one embodiment 300 of a catheter for injecting a viscous medium containing the particles. As depicted, the catheter 300 may include an elongated hollow cylinder having a lumen 304 along the length thereof and a heating element 302, e.g. heating coil, coupled to a power source (not shown in FIG. 3A) via electrical wires 306. In general, the viscous medium to be used in the hyperthermia treatment may be very difficult to force through a lumen 304 at or below the patient body temperature. Typically, the viscosity of the viscous medium decreases as the temperature of the viscous medium increases. The heating element 302 may be operative to generate heat energy so that the temperature of the viscous medium decreases, permitting the viscous medium to flow through the lumen 304 but to revert back to its natural viscous state upon exiting the distal end of the catheter 300.

FIG. 3B shows a schematic longitudinal cross sectional view of another embodiment 310 of a catheter. FIG. 3C shows a schematic transverse cross sectional view of the catheter 310, taken along the line 3C-3C. As depicted, the catheter 310 may include a cavity (or, equivalently, mixing chamber) 315 disposed at the distal end portion of the catheter and at least two lumens 314a, 314b extending from the proximal end of the catheter to the cavity 315. Attached to the inside wall of the cavity are mixing vanes 316 for mixing chemicals flowing through the two lumens. The catheter 312 may be used to generate a highly viscous medium containing the particles by mixing two chemicals. For instance, two constituent chemicals or substrates may have viscosities low enough to allow for flow through the lumens 314a, 314b with small diameters. One of the two constituents may include a prepolymer solution (or, shortly prepolymer) derived from a number of proteins, such as collagen, elastin, fibrin, or the like, or derived from polysaccharides, such as hyaluranic acid, keratin, chondroitin, or the like. The prepolymer can also include synthetic materials, such as peptide, polyalanine, polyactate, or the like. The other constituent may be a cross-linking agent that can include diols, polyols, diacids, diaminespolyamines, or the like.

In one exemplary embodiment, the particles may be homogeneously suspended in the prepolymer solution. Upon mixing of the prepolymer solution with the cross-linking agent in the cavity 315, an in-situ polymerization may occur to result a highly viscous gel like medium containing the particles. The viscous medium may trap the particles near the target site and prevent them from migrating outside the target site.

FIG. 3D shows a schematic longitudinal cross sectional view of yet another embodiment 320 of a catheter of the type to be used in the device 100. FIG. 3E shows a schematic transverse cross sectional view of the catheter 320, taken along the line 3E-3E. As depicted, the catheter 320 may include multiple lumens 324a, 324b and a deployable needle 326 having a proximal tip portion disposed at the distal end of the catheter. A detailed description of the mechanism for operating the needle can be found in U.S. Pat. No. 6,905,476 B2, which is herein incorporated by reference in its entirety.

The needle 326 may be used to inject particle solution in the target tissue that may include, for instance, a weakened sphincter muscle tissue for incontinence treatment and a small tumor tissue to be ablated. Upon injection of the particle solution into the target tissue, it is desirable to clean up excess particles prior to heating. In one exemplary embodiment, the device 100 may be used to pass saline solution (or other suitable cleaning liquid) through one of the lumens 324a, 324b toward the excess particles and draw the contaminated saline solution through the other lumen.

FIG. 3F shows a schematic longitudinal cross sectional view of still another embodiment 330 of a catheter that might be used in the device 100. As depicted, the catheter 330 may include multiple lumens 332a-332c and a balloon 338 formed of inflatable material and in fluid communication with the lumen 332a. The balloon 338 may operate as a sealing mechanism. For example, the distal end portion of the catheter 330 may be inserted into the ductal network 124 and the balloon 338 can be inflated to prevent the liquid (such as particle solution or surfactant) injected into the ductal network 124 from leaking or migrating away from the network (i.e., target region) during treatment. Other types of sealing mechanism, such as a bioabsorbable plug may be used in place of the balloon.

In one exemplary embodiment, the catheter 112 may be formed of material transparent to the external electromagnetic field so that the catheter 112 may remain inserted in the network 124 during treatment. In another exemplary embodiment, the catheter 112 may include tip or shaft formed of radio opaque material for fluoroscopic guidance. In yet another exemplary embodiment, the catheter 112 may include tip or shaft formed of material optimized for ultrasonic imaging systems so as to increase visibility for guidance. In still another exemplary embodiment, the catheter 112 may include extra lumens for tools, optical systems, and fluid/gas inspiration/aspiration. For instance, the tools may include a steering mechanism that is coupled to the steering control 110 for steering the tip or distal end of the catheter so that the practitioner can advance the catheter into intended branches or locations within the patient body. More detailed information about steerable catheter can be found in U.S. Patent 4,960,411, which is herein incorporated by reference in its entirety. It is noted that the catheter 112 may include one or more of the features in FIGS. 3A-3F. For instance, the deployable needle 326 and heating mechanism 302 may be included in the catheter 112 (FIG. 1).

The substrate carrying the particles may have several features. First, as discussed above, the substrate viscosity may be temperature sensitive. Second, the substrate may incorporate two or more constituents that, when mixed, would change the viscosity. Third, the substrate may incorporate an agent to promote coverage of duct wall surfaces and small branches within the ductal network 124. Fourth, the substrate may incorporate a substance that promotes adhesion of particles to target tissue surfaces. Fifth, the substrate may incorporate substances that promote visibility when used with a fluoroscope and/or ultrasonic imaging system.

It should be understood, of course, that the foregoing relates to exemplary embodiments of the invention and that modifications may be made without departing from the spirit and scope of the invention as set forth in the following claims.

What is claimed:

1. A method for delivering particles to a target region in a patient body via a catheter, comprising:
(a) inserting a distal end portion of the catheter into the target region;
(b) directing a first substrate through the catheter to the target region;
(c) drawing the first substrate from the target region through the catheter; and
(d) directing a second substrate containing the particles through the catheter to the target region.

2. A method as recited in claim 1, further comprising:
repeating steps (b)-(c).

3. A method as recited in claim 1, further comprising:
measuring pressures of the first and second substrate during the steps (b)-(d).

4. A method as recited in claim 3, further comprising:
preventing each of the pressures from increasing beyond a preset value.

5. A method as recited in claim 1, wherein the first substrate is same as the second substrate and includes the particles.

6. A method as recited in claim 1, wherein the first substitute includes surfactant.

7. A method as recited in claim 1, wherein the first substrate contains the particles.

8. A method as recited in claim 1, wherein the catheter includes a sealing mechanism operative to prevent the first substrate from leaking or migrating from the target region during one or more of steps (b)-(c).

9. A method as recited in claim 8, wherein the sealing mechanism includes a balloon in fluid communication with a lumen formed in the catheter.

10. A method as recited in claim 1, wherein the catheter includes a heating element, the step of directing a second substrate including heating the second substrate by use of the heating element to reduce the viscosity of the second substrate whereby the second substrate can flow through a lumen of the catheter.

11. A method as recited in claim 1, wherein the catheter includes a cavity disposed at the distal end portion and first and second lumens extending from a proximal end of the catheter to the cavity, the step of directing the second substrate includes:
directing a first constituent containing the particles to the cavity through the first lumen; and
directing a second constituent to the cavity through the second lumen;
whereby the first and second constituents are mixed in the cavity to form the second substrate.

12. A method as recited in claim 11, wherein the first constituent includes a prepolymer containing the particles and the second constituent includes a cross-linking agent whereby mixing of the first and second constituents in the cavity induces polymerization.

13. A method as recited in claim 1, wherein the catheter includes a deployable needle, the step of directing a second substrate includes injecting the second substrate into the target region via the needle.

14. A method as recited in claim 1, wherein the catheter includes a steering mechanism for steering the distal end portion, the step of inserting a distal end portion of the catheter including advancing the distal end portion toward the target region by use of the steering mechanism.

15. An apparatus for delivering substrate to a target region in a patient body, comprising:
a hub;
a catheter having a proximal end coupled to and in fluid communication with the hub and a distal end to be inserted into the target region;
means for directing substrate to the target region, the means being coupled to the hub and in fluid communication with the catheter;
a pressure sensor for measuring a pressure of the substrate and generating a signal commensurate with the pressure; and
a pressure display for displaying the pressure in response to the signal.

16. An apparatus as recited in claim 15, wherein the means for directing the substrate includes at least one syringe containing the substrate.

17. An apparatus as recited in claim 15, further comprising:
a selection valve for controlling a flow of substrate from the hub to the catheter.

18. An apparatus as recited in claim 15, wherein the catheter includes a heating element for heating the substrate.

19. An apparatus as recited in claim 15, wherein the catheter includes a cavity formed near the distal end and first and second lumens extending from the proximal end to the cavity and wherein the means for directing substrate includes first and second syringes that are respectively in fluid communication with the first and second lumens.

20. An apparatus as recited in claim 19, wherein the first syringe contains a prepolymer including particles and the second syringe contains a cross-linking agent whereby mixing of the first and second constituents in the cavity induces polymerization.

21. An apparatus as recited in claim 15, wherein the catheter includes a deployable needle disposed at the distal end and operative to inject the substrate into the target region.

22. An apparatus as recited in claim 15, wherein the catheter includes a steering mechanism for steering a distal end portion thereof.

23. An apparatus as recited in claim 15, wherein the catheter include a sealing mechanism for preventing the substrate from leaking or migrating from the target region and in fluid communication with a lumen formed in the catheter.

24. An apparatus as recited in claim 15, further comprising:
a pump motor for actuating the means for directing substrate; and
a microcontroller for controlling the pump motor.

25. An apparatus as recited in claim 24, wherein the microcontroller is responsive to the signal.

26. An apparatus as recited in claim 15, wherein the hub includes a safety pressure valve for preventing the pressure from increasing beyond a preset level.
